(19) United States
(12) Patent Application Publication
(10) Pub. No.: US 2014/0052203 A1
(43) Pub. Date: Feb. 20, 2014

(54) MRI COMPATIBLE IMPLANTABLE ELECTRONIC MEDICAL LEAD

(71) Applicant: Kenergy, Inc., Mequon, WI (US)

(72) Inventor: Cherik Bulkes, Sussex, WI (US)

(73) Assignee: Kenergy, Inc., Mequon, WI (US)

(21) Appl. No.: 13/967,701

(22) Filed: Aug. 15, 2013

Related U.S. Application Data
(60) Provisional application No. 61/683,539, filed on Aug. 15, 2012.

(51) Int. Cl. A61N 1/375

(52) U.S. Cl. CPC A61N 1/375
USPC 607/5; 607/116; 607/9

(57) ABSTRACT

An implantable electrical lead that, upon implantation in an animal, is biocompatible and compatible with a magnetic resonance imaging scanner. The upon implantation in an animal has a body of dielectric material with a plurality of lumens and a plurality of insulated conductive helical coils embedded in one or more layers of dielectric material and placed within the plurality of lumens. Each helical coil is formed by one or more conductive wires having a predefined and controlled pitch and diameter. A layer of dielectric material separates the plurality of lumens, wherein the separation distance and properties of the dielectric material create a high impedance at the Larmor frequency of the magnetic resonance imaging scanner. A mechanically flexible, biocompatible layer forms an external layer of the electrical lead and is adapted to contact bodily tissue and bodily fluids of the animal.

### CROSS-REFERENCE TO RELATED APPLICATIONS

This application claims benefit of U.S. Patent Provisional Patent Application No. 61/683,539 filed on Aug. 15, 2012.

### STATEMENT CONCERNING FEDERALLY SPONSORED RESEARCH OR DEVELOPMENT

Not Applicable

### BACKGROUND OF THE INVENTION

1. Field of the Invention

The present invention relates to implantable electronic medical leads, such as those used with cardiac pacemakers and defibrillators for example, for stimulating the tissue of an animal for therapeutic purposes, and more particularly to such implantable medical leads that are compatible with magnetic resonance imaging.

2. Description of the Related Art

Numerous medical conditions, such as cardiac and neurological dysfunctions, are treated by an implanted electronic device which provides electrical stimulation to the affected tissue of the animal. These devices have a plurality of metal components, including the generator case and wire leads extending from the case to electrodes in contact with the tissue to be stimulated or monitored.

Magnetic resonance imaging (MRI) is commonly employed to view internal organs of medical patients. To create an image, the patient is placed into very strong static and varying magnetic and radio frequency (RF) fields and thus MRI generally is prohibited for patients with implanted ferromagnetic and or electrically conductive objects. Although it is feasible to minimize and even eliminate the use of ferromagnetic materials in implanted apparatus, devices, such as cardiac pacemakers and defibrillators, require electrically conductive components that are affected by the fields produced by an MRI scanner.

There is a need to make implanted devices MRI compatible so that this imaging modality can be used with patients having those devices. There are several reasons for achieving this goal. First, incompatible implant components induce susceptibility difference, which destroys DC magnetic field homogeneity, thereby affecting the imaging performance of the magnetic resonance scanner. Second, conductive materials present an opportunity for eddy currents to form, which currents generate heat that adversely affects patient safety and degrade the scanner performance by field distortion. Third, the MRI fields may ruin the implanted device. Fourth, the incompatible implant material can potentially cause serious internal injuries to the patient.

The issue of MRI interaction with electronics of an implanted device has to be considered in an integrated fashion to provide compatibility. Table 1 shows combinations of interactions that are briefly discussed hereinafter.

TABLE 1
Interactions of Factors Influencing MRI Compatibility
of an Implanted Device or Component

| | Patient | Effect on the | Effect on the |
|---|---|---|---|
| | Safety | Implanted Device | MR Image |
| DC Magnetic Fields | I | II | III |
| Gradient Magnetic Fields | IV | V | VI |
| Radio Frequency Fields | VII | VIII | IX |

I. Any ferromagnetic material inside the implanted device exposed to the MRI fields experiences a force and a torque, the amount of which depends on the shape, dimensions, and amount of ferromagnetic material. The forces are greatest in areas where there is a gradient in the magnetic field, e.g. upon entering a MRI system. Obviously the surrounding tissue adjacent the implantable device will be damaged in this case and the health of the patient will be compromised. In addition, metallic components can become hot and burn the patient.

II. Due to MRI field induced torque and movement of the implanted device, its components may become disconnected making the device inoperable. Ferrites and other ferromagnetic material in transformer cores, inductors and other electronic components become saturated, thereby jeopardizing the function of the medical device. Heating causes electronic components to operate out of specification.

III. The homogeneity of the magnetic resonance imager's DC magnetic field will be distorted, destroying spectral resolution and geometric uniformity of the image. The inhomogeneous field also results in rapid de-phasing of the signal inside the excited volume of the patient. The resultant image shows a distorted view of the patient's anatomy.

Even if the implanted device does not contain any ferromagnetic materials, the magnetic susceptibility of the device may be different than that of the surrounding tissue, giving rise to local distortion and signal dropouts in the image, close to the device. This is especially true for pulse sequences that are sensitive to phase, like echo planar imaging

IV. Switching field gradients create large eddy currents, at frequencies up to a few kilohertz, in the metallic housing of an implantable device and any metallic part that forms a loop, such as cables forming a loop. These eddy currents make the device move with the same frequency as the leading and trailing edges of gradient pulses. This movement can be unsafe for the surrounding tissue. The associated eddy current pattern creates local pulsating E-fields, in addition to the E-field generated by the MRI scanner's gradient coil, which can stimulate the patient's nerves. Resultant muscle twitching can be so intense as to be painful.

V. The eddy currents may be strong enough to damage electronic circuits and destroy the implanted device. The pulsating forces on the device may disconnect components.

VI. The eddy currents affect the rise time of the MRI gradient pulses, and therefore affect the minimum obtainable echo time, necessary for many pulse sequences. The eddy currents also locally distort the linearity of the gradient fields and de-phase the spin system, resulting in image distortion and signal dropouts. Phase and frequency encoding of the signal strongly depends on the linearity of the gradients.

VII. The RF field interacts with any metallic part in the device, be it either in the form of a loop, which results in B-field coupling, or a straight conductor, which results in E-field coupling. The B-field component of the RF field can induce currents and voltages in conducting loops. The amplitude depends on the impedance of the loop at the RF frequency, and the size of the loop. An example may be two coaxial cables that form a loop together. Such a loop may have high impedance at DC due to the insulating outer shell of the coax, but the distance between the cables at the crossover point may be equivalent to just the right amount of capacitance to make the loop resonant at the RF frequency.

The E-field component of the RF field will induce voltages and currents in conductors, such as a single cable for example. The amplitude of the induced voltages and currents depends on the phase length of the conductor, or path, at the associated radio frequency.

The induced voltages and currents create locally very strong E-fields, in particular at the ends of the electrical, which can burn the patient.

Non-metallic implantable devices do not have these issues, but can still distort the uniformity of the RF field if the permittivity of the device is different than that of the surrounding tissue. This distortion is especially strong at radio frequencies above 100 MHz.

VIII. Localized high voltages and currents in the medical device may cause components to fail either due to high voltage arcing or due to dissipated power and heat. This includes connections that become unsoldered due to the heat. The device may generate pulsed voltages at unwanted times and locations in the leads of a cardiac pacemaker.

IX. Local distortion of the uniformity of the B-field component of the RF field will give rise to flip angle variation and creates contrast and signal-to-noise ratio (SNR) inhomogeneity. The specific absorption rate, which is defined as the RF power absorbed per unit of mass of an object, can exceed legal limits set by governmental regulatory agencies. If the specific absorption rate exceeds legal limits, images cannot be made using magnetic resonance scanners.

From a fundamental physical perspective, it is useful to examine the conductivity of wires at high frequencies of MRI. As frequencies increase, conduction begins to move from an equal distribution through the conductor cross section toward existence almost exclusively near the surface. Depending on the conductor bulk resistivity, at sufficiently high frequency all the RF current is flowing within a very small thickness at the surface. Lower bulk resistivities result in shallower skin depths.

For a solid wire, the electric current concentrates on the outer surface. For this reason, when skin depth is shallow, the solid conductor can be replaced with a hollow tube with no perceivable loss of performance. Choice of a plating material can degrade performance (increase attenuation) if its bulk resistivity is greater than that of the body of the wire. If such a conductor is placed inside the E field of an MRI RF transmit coil, there will be RF energy deposition in the tissue surrounding the wire resulting in elevated temperatures that may result in physical injury to the patient.

Therefore it is desirable to make electrical leads, which connect implanted medical devices to the electrodes for stimulation therapy and/or electrical sensing, MRI compatible.

Various types of multi-lumen implantable leads have been suggested in prior art, however, these designs contain straight wires, straight stranded cables or very small coiled wires, which are not suitable for MRI compatibility. As discussed above, conductors, whether they are solid or stranded construction, may result in high E-fields at the conductor end or can be prone to hotspots partway along the lead body if resonance occurs from the induced MRI scanner's RF energy, which in turn result in elevated tissue temperatures that can be potentially injurious to the patient. Small coils that can be easily distorted when compressed, stretched, or flexed, as would occur in vivo as a result of (for example) movement or trauma, cannot maintain the close dimensional and electrical parameters that are necessary for MRI compatibility.

Various approaches to MRI compatibility of implantable leads have also been suggested in prior art that are aimed at reducing lead heating and associated tissue damage. For example, several inventors have proposed incorporating a variety of inductive and capacitive elements (i.e. resonant tanks, chokes, bandstop filters) to reduce resonance in the lead at selective frequencies (see U.S. Pat. No. 8,433,421, U.S. Pat. No. 8,463,375). Others propose using an electrically conductive sleeve to shield the underlying helix and also to provide a lossy layer to discourage resonance within the lead. More recently, methods have been proposed to incorporate switching circuits that open the lead and divert current to through a dissipating surface in the presence of high electromagnetic fields (see U.S. Pat. No. 8,457,760 and U.S. Pat. No. 8,494,646), or thermally sensitive materials that transition to a high impedance state when temperatures exceed safe limits (see U.S. Pat. No. 8,478,421).

The present invention uses a novel approach of optimizing design parameters (e.g. coil inductance, interwinding capacitance, distance between conductive helical coils, conductivity and permittivity of materials, and capacitance of the collective helical structure to the nearby body tissue and or fluids) to minimize the energy that can be absorbed, thereby reducing or preventing self-resonant modes from setting up within the lead. This approach presents the added benefits, by means of reduced RF energy absorption, of preventing unwanted stimulations and/or damage to electronic components within the implanted device, while enabling diagnostic quality imaging by means of allowing a wider range of the more RF intense protocols over a broad range of imaging field strengths.

A multi-lumen approach to MRI compatible lead design has also been proposed in which a tubular, multi-lumen insulator is coiled following insertion of individual conductors (See U.S. Application 2013/0184550A1). The present invention differs from this approach by utilizing conductive helical coils inserted into a straight multi-lumen tubular structure to achieve MRI compatibility. This enables closer control of the relative position of the conductors which is essential for MRI safe operation. Consideration must also be given to the resultant final diameter of the structure and the resistance of the conductors to enable the intended application. For example, for a defibrillator, resistances of 2 to 5 ohms or less are required; slightly higher for cardioverters. Nevertheless this requirement and desirable lead diameter (usually nine French or less) can be met with the proposed solution, while difficult if not impractical with the other solutions.

### SUMMARY OF THE INVENTION

This invention is directed toward an implantable biocompatible electrical lead that is also compatible with being safely scanned in a magnetic resonance imaging scanner for the purpose of medical diagnostic quality imaging using commonly used protocols. The implantable electrical lead comprises a tubular length of dielectric material with a plurality of lumens extending over its entire length (hereafter referred to as the “multi-lumen body structure”). The dielectric material, size of the lumens, distance between lumens and outer thickness of the dielectric layer are closely controlled and are selected based on minimizing or suppressing the buildup of standing waves in the lead when exposed to the electromagnetic fields of an MRI scanner. A plurality of insulated conductive helical coils consisting of one or more conductive wires and embedded in one or more layers of dielectric material are placed within the multi-lumen body structure. The diameters of the conductive wires, the diameters of the helical coils, the lengths of the helical coils, the directions of winding, the winding pitch, the spacing between groups of conductors, and the dielectric material and the thickness of layers are closely controlled and are selected based on minimizing or suppressing the buildup of standing waves in the lead when exposed to the electromagnetic fields of an MRI scanner. Although the solution is intended to be non-resonant or broad band, optimization for higher field strengths of, for example, 1.5 T through 3.0 T, is desirable, as these are the prominent field strength systems in clinical use. This optimization is accomplished by choice of inductance, interwinding capacitance and helical coil structure to tissue coupling, such that the formation of standing waves on the lead is discouraged. The helical coils, although embedded in layers themselves for inductance and interwinding capacity stability, are separate from the multi-lumen body structure and one or more of the helical coils are free to move longitudinally and rotationally within their respective lumens of the multi luminal structure. In one embodiment of this invention, the winding direction of all helical coils is the same. In another embodiment of this invention, the winding direction of the helical coils varies.

In one embodiment, the lead is part of an electrical stimulation system. In another embodiment, the lead is part of a cardiac pacing system. In yet another embodiment, the lead is part of a cardiac defibrillation system, or cardioverter system.

The invention may utilize any of a variety of pace/sense electrodes that are currently available or may become available. The invention may also include a passive or active fixation mechanism at the distal end, which is secured into the tissue to facilitate positioning of the electrode(s). In a preferred embodiment, an active fixation type device is used which also functions as a pace/sense electrode.

The invention also contemplates an implantable defibrillation lead compatible with being safely scanned in a magnetic resonance imaging scanner for the purpose of medical diagnostic quality imaging. The implantable defibrillation lead comprises a plurality of helical coils placed within a multi-lumen body structure wherein at least one of insulated conductive coils come out of the lead body without insulation and is electrically connected to a non-insulated conductive coil.

### BRIEF DESCRIPTION OF DRAWINGS

FIG. 1 is a cross-sectional view of an exemplary multi-lumen body structure.

FIG. 2 is an isometric view of an exemplary quad-lumen lead assembly.

FIG. 3 is a longitudinal cross-section of a bifilar conductive helical coil assembly.

FIG. 4 is a longitudinal cross-section of a monofilar conductive helical coil assembly.

FIGS. 5 and 6 are longitudinal cross-sections of an MRI compatible defibrillator lead assembly.

### DETAILED DESCRIPTION OF THE INVENTION

The present technique for magnetic resonance compatibility of an implanted electronic medical lead considers several effects of direct current (DC) magnetic fields, gradient magnetic fields, and RF fields on patient safety, the implanted lead and the MRI scanner. As a consequence, the medical lead described herein incorporates one or more mechanisms that offer high impedance to currents induced by the MRI electromagnetic fields or prevent such currents from forming in the first place. In addition to using non-ferromagnetic components which have a magnetic susceptibility close to that of the surrounding tissue, these mechanisms comprise a multi-lumen body structure and multiple conductive helical coils.

### Multi-Lumen Body Structure

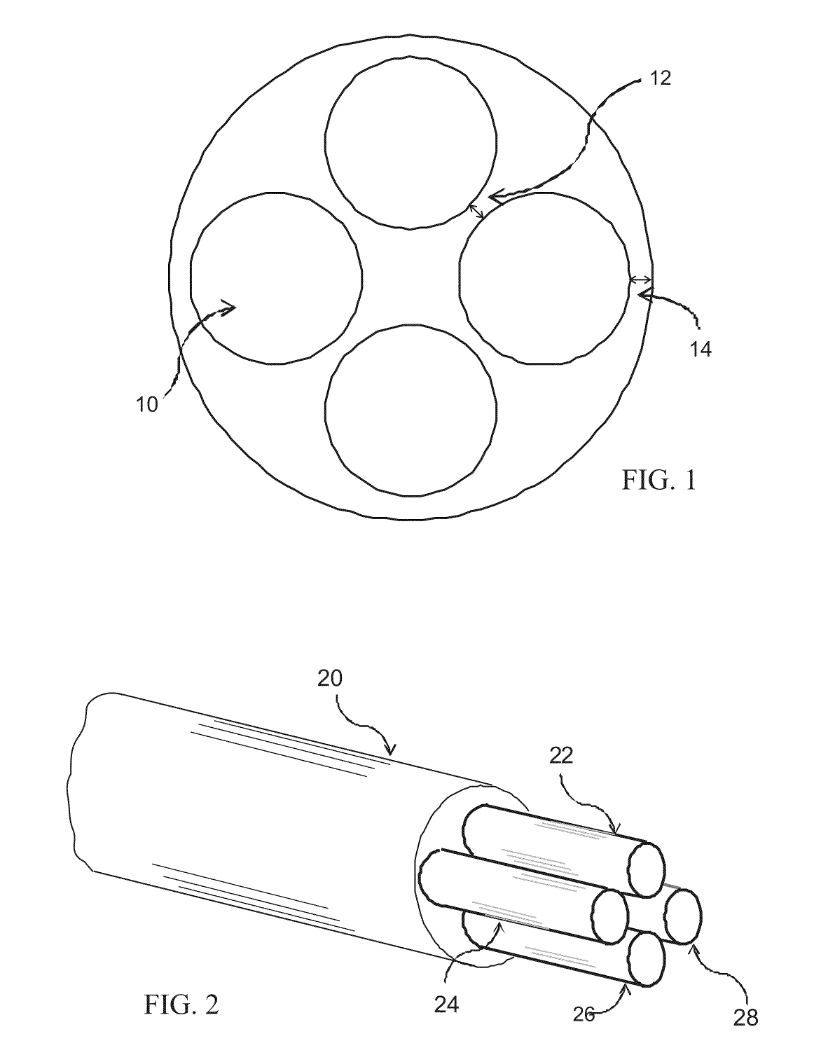

The multi-lumen body structure comprises a length of tubular dielectric material with a plurality of lumens extending over its entire length. A cross section of this structure is shown in FIG. 1. The size of the lumens 10, the distance 12 between adjacent lumens, the outer thickness of the dielectric layer 14 and the dielectric material are closely controlled and are selected based on minimizing or suppressing the buildup of standing waves in the lead when exposed to the electromagnetic fields of an MRI scanner.

FIG. 2 illustrates one embodiment of the current invention in which a multi-lumen body structure 20 houses four helical coils 22, 24, 26, 28. The helical coils may vary in terms of wire diameter, wire material, coil diameter, number of conductors, direction of wind, winding pitch, spacing between groups of conductors, and overall length; or the characteristics of helical coils may be varied selectively depending on the application for which the lead is to be used.

A first example of a multi-lumen lead is a quad lumen lead using helical coils of various pitches and diameters. Note that for the purpose of clarity, a dual bifilar/dual monofilar is discussed here, but other combinations are possible as well. A first lumen containing bifilar helical coil 22 is separated from a second lumen containing monofilar helical coil 24 by a suitable dielectric material (e.g., polyurethane). A third lumen contains a second bifilar helical coil 26 and a fourth lumen contains a second monofilar helical coil 28. Each helical coil is insulated from the other helical coils by a suitable dielectric material (e.g. polyurethane).

The potential resonant length of the lead and its component helical coils is a function of a wavelength of interest which is determined by the velocity of the electromagnetic wave in the animal tissue divided by the frequency of the electromagnetic wave. The velocity is the inverse of the square root of the product of permittivity and permeability of the tissue. To minimize the opportunity for lead body resonance, the lead length is preferably longer than half of the wavelength of interest for a 1.5 Tesla (T) MRI scanner operating at 64 MHz or a 3.0 T MRI scanner operating at 127.7 MHz. The same applies to any other frequency, although 1.5 T and 3.0 T are the primary field strengths for clinical use. In an embodiment, leads are designed to be a low quality or heavily dampened antenna at 64 MHz for a 1.5 T MRI scanner or at 127.7 MHz for a 3 T MRI scanner. In addition, the half wavelength transmission line is terminated on both ends, with potentially high E-field concentration on these ends. However, the E-field concentration is also a function of the tip diameter, i.e. a smaller radius tip will yield a higher local E-field than a larger radius tip. The proximal end of the lead terminates in the generator, which for RF is terminated in the tissue, but with a much larger overall radius, which sufficiently limits the local E-field below a level that poses a heating risk to the patient.

In some embodiments that are contemplated in the current invention, special considerations need to be taken to ensure MRI compatibility. These considerations may include avoiding loops in the lead in any of the potential routing paths unless the distance at the crossover point between the two ends of the lead forming a loop, is larger than approximately ten lead diameters.

### Helical Coils

The overall length of the helical coils, the diameter of the wire, the helical diameter, the winding pitch, the spacing between groups of conductors, and the dielectric material and the thickness of layers are selected to provide high impedance to radio frequency currents induced in the cable while presenting low impedance to direct current of stimulation pulses produced by the medical device. Such helical coils provide sufficiently high impedance, reactance and/or resistance, to prevent induced current from forming during MRI radio frequency pulses in the 3-150 MHz range.

The parameters that characterize the electrical characteristics of the helical coils include winding pitch, turn to turn conductor distance, coaxial radial spacing, permittivity of dielectric and thickness of insulating layers. Having more turns per centimeter will increase inductance but also interwinding or parasitic capacitance. Increasing turn to turn spacing will decrease parasitic capacitance. The electrical and dimensional parameters of each helical coil must be closely controlled over its entire length in order to minimize the induced voltages and currents that can cause localized heating and/or image distortion. This is accomplished by embedding the helical coils in one or more layers of dielectric material that are fused together, permanently securing the conductive coil and preserving the helix pitch, the helical diameter and the spacing between groups of conductors.

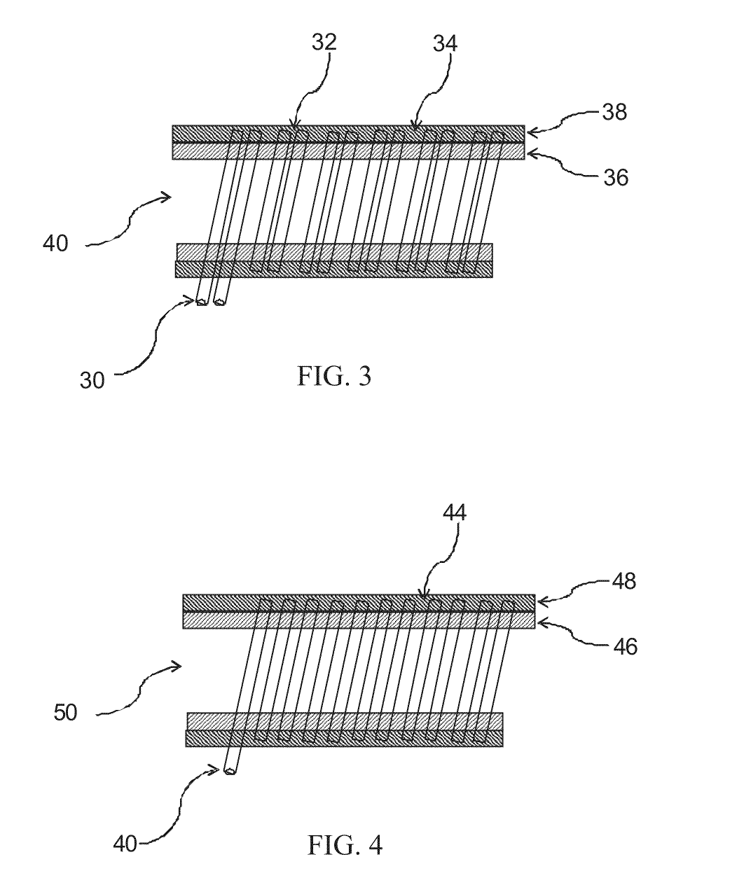

FIG. 3 illustrates an example of a bifilar helical coil construction in which a pair of conductors 30 is wound in such a way as to control the spacing between the conductors 32 and the spacing between the conductor pairs 34. The winding pitch, the spacing between conductors and the helical diameter together determine the interwinding capacitance. This capacitance, along with the inductance from the windings, form an LC combination with a resonant frequency. This resonant frequency is not allowed to reach low enough (e.g. 128 MHz for 3.0 T MRI) to allow the lead to become self-resonant. Self-resonance could lead to excessive EM field concentration around the lead and high E-field amplitudes at the ends of the lead, in turn causing high peak E-field strength at the distal tip, leading to potential RF burns. The helical coils may be wound in a clockwise (CW) direction or a counter-clockwise (CCW) direction. The helical coil is covered by an insulator/biocompatible material (e.g. Kapton or polyurethane) to prevent the external surface from coming in contact with body fluids (e.g., blood).

In one embodiment, the conductors are embedded between multiple layers of insulating material 36, 38 which is reflowed around the coiled conductors. This design not only improves the structural integrity of the helical coil but also provides ample space for an air core 40 for allowing insertion of a guide wire. However, care should be taken in this design to prevent any body fluid from entering at the ends of the helical coil. It should be noted that electrical properties of the helical coil are dependent on the inner insulation thickness as well as the permittivity of the insulating material. Further it should be noted that the inductance of the helical coil increases with increased diameter of the helix of bifilar (or multifilar) conductors. In practice, however, this diameter cannot be arbitrarily varied since it is fixed due to the restriction imposed on the dimensions of an intravascular lead structure

A second example of a helical coil may have a monofilar configuration, as shown in FIG. 4 in which a single conductor is wound in such a way as to control the spacing between the turns 44. The winding pitch and the helical diameter together determine the interwinding capacitance. This capacitance, along with the inductance from the windings, form an LC combination with a resonant frequency. This resonant frequency is not allowed to reach low enough (e.g. 128 MHz for 3.0 T MRI) to allow the lead to become self-resonant. Self-resonance could lead to excessive EM field concentration around the lead and high E-field amplitudes at the ends of the helical coil, in turn causing high peak E-field strength at the distal tip, leading to potential RF burns. The helical coils may be wound in a clockwise (CW) direction or a counter-clockwise (CCW) direction. The helical coil is covered by an insulator/biocompatible material (e.g. Kapton or polyurethane) to prevent the external surface from coming in contact with body fluids (e.g., blood).

In one embodiment, the conductors are embedded between multiple layers of insulating material 46, 48 which is reflowed around the coiled conductors. This design not only improves the structural integrity of the helical coil but also provides ample space for an air core 50 for allowing insertion of a guide wire. However, care should be taken in this design to prevent any body fluid from entering at the ends of the helical coil. It should be noted that electrical properties of the helical coil are dependent on the inner insulation thickness as well as the permittivity of the insulating material. Further it should be noted that the inductance of the helical coil increases with increased diameter of the conductive helix. In practice, however, this diameter cannot be arbitrarily varied since it is fixed due to the restriction imposed on the dimensions of an intravascular lead structure.

When assembled within the overall multi-lumen body structure there can be a combination of mono and multi-filar helical coils to support the various stimulation and/or sensing functions traditionally found in pacing and defibrillation applications. Specifically sense and pace circuits are required as are one or more shocking coil conductors. Multiple parallel filars may also be connected to a single electrode to match the electrical requirements of the generator system and/or electrode application.

Various combinations of mono or multi filar conductor combinations along with mono and multi luminal structures are possible to accommodate the conductor pathways.

### Quadlumen Configuration:

Quadlumen configuration: This can accommodate up to two shocking circuits and two or more pace and sense circuits.

### Trilumen Configuration:

This can accommodate one or two shocking circuits and one pace/sense circuit conductor pair.

### Bilumen Configuration:

This can accommodate one shocking circuit and one pace/sense circuit conductor pair.

### MRI Compatible Defibrillation Lead:

For an MR compatible defibrillation (ICD) lead, multiple circuits are required. Typical configurations include a single or two shocking coil circuits. These circuits will carry the discharge current required for defibrillation and can be at potentials as high as 700V or more and need to be sufficiently electrically isolated from the pace and sense circuits. The pace and sense circuit usually share the same pair of conductors, one for the distal tip electrode, and another for the ring electrode. In some cases multiple ring electrodes can be used for additional stimulation site flexibility. To ensure sufficient isolation and mechanical stability, multi luminal designs are used.

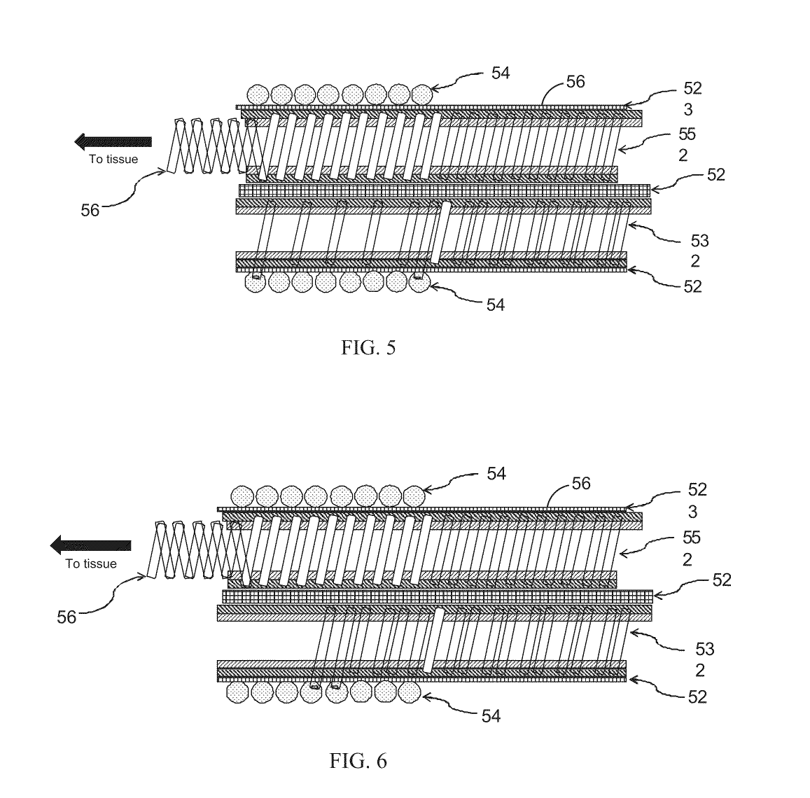

Referring to FIG. 5, a defibrillation lead is comprised of multiple helical coils covered within a quad-lumen body structure 52. In an exemplary case of a two shocking coil defibrillator configuration, the insulated conductors of a helical coil 53 exit the lead body without insulation and are connected to a shocking coil either at both ends (FIG. 5) or in the center of the coil (FIG. 6).

Two additional helical coils (not shown) are for cardiac pacing. The end termini are connected to the pacing electrodes (not shown). If the inner insulated conductor for pacing is more than one-eighth of a wavelength of the MRI scanner in contact with the body fluid or tissue for pacing, then the medium conducting coating covers the surface of the helical coil followed by an outer insulating layer. The helical coil 55 is present throughout the lead and is terminated with an anchoring component 56 which helps in the anchoring of the lead. The anchoring component is made up of an MRI compatible material described earlier. An electrically conductive layer 54 is placed around at least one or more of the conductive helical coils and the body structure 52.

### An Integrated Approach to MRI Compatibility:

An integrated approach to MRI compatibility involves a lead assembly simultaneously satisfying the following conditions: (a) there are no susceptibility effects from materials used for the lead construction to avoid image artifacts; (b) the materials used are non-magnetizable to avoid image artifacts; (c) the lead design minimizes buildup of induced common mode currents while the lead is being exposed to the MRI RF field; (d) the lead design avoids formation low frequency (0.001 kHz-10 kHz) conductive loops so that the lead structure is unaffected by the gradient field; (e) the lead is flexible enough to be usable for long term bio implant use, for example, in electrical stimulating devices such as cardiac pacemakers, defibrillators, and nerve stimulators; and (f) the lead is biocompatible such that it does not promote or cause any adverse reaction to the user. Thus, a key aspect of the invention is achieving simultaneous electrical, mechanical and biological compatibility.

### Achieving Electrical Compatibility:

Minimizing the buildup of induced common mode currents involves reducing the ability of the lead to be an antenna, i.e. a receptacle for RF energy. The electrical compatibility of individual helical coils is achieved as described above. However, placing multiple conductive helical coils in close proximity within the multi-lumen body structure creates a transmission line topology in which the resultant circuit resembles a chained LC network with the primary inductance being in the helical coils and the primary capacitance being between the helical coils. Therefore, to prevent the overall lead assembly from becoming an antenna, it is also necessary to carefully control selection of the dielectric material, the thickness of the insulating layers and the positioning of the helical coils within the multi-lumen body structure.

Since the surrounding tissue is capacitively coupled to the lead via the intermediate insulation between the helical coils and the tissue, managing this distance controls the amount of energy that is dissipated along the length of the lead to minimize build-up of energy at the lead ends.

The effectiveness of the antenna can be reduced further by the addition of an electrically conductive layer, either to the individual helical coils or to the multi-lumen body structure. This will cause a damping of its resonance and act as a shield to reduce the amount of energy it can potentially absorb. The conductivity must be low enough to avoid the conductive layer itself from forming standing waves, but high enough to provide damping. An example of this is the use of a graphite layer with a conductivity in the range of 1.00 to 10⁴ Siemens per meter. The electrically conductive layer 56 in FIGS. 5 and 6 can comprise a material that has electrically conductive, non-magnetizable particles in physical contact with each other.

Since focal spots in the E-field can be created by concentration of E-field, such as at tips or ends of wires or components, any sharp edge or point is avoided.

### Achieving Mechanical and Biological Compatibility:

The mechanical and biological compatibility is obtained using the steps described below: First, the flexibility of the lead is required to allow for the lead to follow the body and intra-organic movements, without impediment. Second, the fatigue resistance is essential for many applications, for example, in a cardiac apex application, the lead end would flex with each heartbeat. Third, considerations are given to satisfy both flexibility and fatigue resistance simultaneously in addition to providing biocompatibility. Polyurethane materials are used for the lead body to meet all the three criteria. In addition, the conductor material is chosen from the well-known alloys, for example, MP35, stainless steel, which are specifically designed to have a very high fatigue resistance and tensile strength against breakage.

The foregoing description was primarily directed to one or more embodiments of the invention. Although some attention has been given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

I claim:

1. An implantable electrical lead that is biocompatible upon implantation in an animal and compatible with being safely scanned in a magnetic resonance imaging (MRI) scanner for a purpose of diagnostic quality imaging, using common standard imaging protocols, such as spin echo, fast spin echo, gradient recalled echo, echo planar imaging, steady state free precession and comparable protocols, wherein the magnetic resonance imaging scanner is responsive to signals at a Larmor frequency, said implantable electrical lead comprising:
a body of dielectric material with a plurality of lumens extending over an entire length of the body;
a plurality of insulated conductive helical coils comprising one or more conductive wires having a predefined and controlled pitch and diameter, embedded in one or more layers of dielectric material and placed within the plurality of lumens;
a layer of dielectric material separating the plurality of lumens by a distance, wherein the distance and properties of the dielectric material create a high impedance at the Larmor frequency; and
a mechanically flexible, biocompatible layer forming an external layer of the implantable electrical lead and adapted to contact at least one of bodily tissue or bodily fluids of the animal.

2. The implantable electrical lead as recited in claim 1 further comprising one or more electrodes connected to one or more insulated conductive helical coils for applying electric current to stimulate the animal.

3. The implantable electrical lead as recited in claim 2 further comprising one or more electrodes connected to one or more insulated conductive helical coils for applying electric current to the animal for cardiac pacing.

4. The implantable electrical lead as recited in claim 2 further comprising one or more electrodes connected to one or more insulated conductive helical coils for applying electric current to the animal to perform cardiac defibrillation on the animal.

5. The implantable electrical lead as recited in claim 1 wherein the dielectric material, size of the lumens, distance between lumens and outer thickness of the dielectric layer, which form the body, are closely controlled and are selected based on minimizing or suppressing buildup of standing waves in the electrical lead when exposed to electromagnetic fields of an MRI scanner.

6. The implantable electrical lead as recited in claim 1 wherein a first plurality of insulated conductive helical coils are wound in a first direction and a second plurality of insulated conductive helical coils are wound in a different second direction.

7. The implantable electrical lead as recited in claim 1 wherein a first plurality of insulated conductive helical coils are wound in a first direction and a second plurality of insulated conductive helical coils are wound in the first direction.

8. The implantable electrical lead as recited in claim 1 wherein one or more of the insulated conductive helical coils are separate from the body and are free to move longitudinally and rotationally within their respective lumens.

9. The implantable electrical lead as recited in claim 1 wherein the plurality of insulated conductive helical coils are a combination of monofilar and multi-filar helical coils.

10. The implantable electrical lead as recited in claim 1 wherein the Larmor frequency is one of approximately 64 MHz or approximately 128 MHz.

11. The implantable electrical lead as recited in claim 1 wherein the high impedance created by properties of the dielectric material, size of the lumens, distance between lumens and outer thickness of the dielectric layer prevent currents from forming in the implantable electrical lead due to electromagnetic fields of the magnetic resonance imaging scanner.

12. The implantable electrical lead as recited in claim 1 further comprising an electrically conductive layer placed around at least one or more of the conductive helical coils and the body.

13. The implantable electrical lead as recited in claim 12 wherein the electrically conductive layer has a conductivity between 1.00 and 10⁴ Siemens per meter.

14. The implantable stimulation lead as recited in claim 1 wherein the plurality of insulated conductive helical coils extends from one end of the implantable electrical lead to another end.
